(19) United States
(12) Patent Application Publication
(10) Pub. No.: US 2022/0323452 A1
(43) Pub. Date: Oct. 13, 2022

(54) METHODS AND COMPOSITIONS FOR INHIBITING GAPDH

(71) Applicant: Geistlich Pharma AG, Wolhusen (CH)

(72) Inventors: Hanns Moehler, Maennendorf (CH); James C. Costin, Phoenixville, PA (US); Thomas Mueller, Wolhusen (CH)

(73) Assignee: Geistlich Pharma AG, Wolhusen (CH)

(21) Appl. No.: 17/612,953

(22) PCT Filed: May 21, 2020
(86) PCT No.: PCT/IB2020/054853

§ 371 Date: Nov. 19, 2021

Related U.S. Application Data
(60) Provisional application No. 62/851,436, filed on May 22, 2019.

(51) Int. Cl. A61K 31/549; A61K 31/18; A61K 31/4166; A61K 31/53; A61K 31/17; A61K 31/205; A61K 49/00

(52) U.S. Cl. CPC A61K 31/549; A61K 31/18; A61K 31/4166; A61K 31/53; A61K 31/17; A61K 31/205; A61K 49/0004

(57) ABSTRACT

A method of inhibiting GAPDH with N-methylol transfer agents and/or related compounds.

### FIELD OF THE DISCLOSURE

This disclosure relates to compositions and methods for treating, inhibiting, preventing or reducing disorders and diseases in a subject by administering one or more anti-GAPDH agents of the present disclosure.

### BACKGROUND

Glyceraldehyde-3-phosphate dehydrogenase (GAPDH) is involved in a complex array of cellular pathways. In addition to the cytoplasm where the majority of GAPDH is located under the basal condition, GAPDH is also found in the particulate fractions, such as the nucleus, the mitochondria, and the small vesicular fractions. When cells are exposed to various stressors, dynamic subcellular re-distribution of GAPDH occurs. In particular, GAPDH is an important enzyme for energy metabolism and the production of ATP and pyruvate through aerobic glycolysis in the cytoplasm. Although increased GAPDH gene expression and enzymatic function is associated with cell proliferation and tumorigenesis, conditions such as oxidative stress impair GAPDH catalytic activity and lead to cellular aging and apoptosis. A variety of interacting partners for GAPDH, including proteins, various RNA species and telomeric DNA have been identified, yet the mechanisms underlying the effects of GAPDH on cellular proliferation remain unclear.

Several studies indicate that GAPDH has pleiotropic functions independent of its canonical role in glycolysis. The GAPDH functional diversity is mainly due to post-translational modifications in different amino acid residues or due to protein-protein interactions altering its localization from cytosol to nucleus, mitochondria or extracellular microenvironment. Non-glycolytic functions of GAPDH include the regulation of cell death, autophagy, DNA repair and RNA export, and they are observed in physiological and pathological conditions as cancer and neurodegenerative disorders.

The oligomeric state of GAPDH and its propensity to aggregate is mainly dependent on various signal molecules. The redox sensitive cysteine residues of the enzyme, which includes Cys-152 in the active site, are also target of reactive oxygen species (ROS) or reactive nitrogen species (RNS) and, consequently, GAPDH aggregation is influenced by several other stimuli inducing cellular oxidative/nitrosative stresses. Besides cancer, the functional versatility of this enzyme determines that GAPDH alteration is involved in several other diseases especially neurodegenerative disorders such as Alzheimer's disease (AD), Parkinson's disease (PD) and Huntington's disease (HD).

The non-glycolytic roles of GAPDH include physio-pathological functions such as regulation of gene expression, DNA repair and replication, neurodegeneration, pathogenesis, virulence in bacteria, tubular bundling, protein-protein interactions, RNA export, as well as apoptosis and autophagy. For instance, it has been discovered that GAPDH acts as a key component of the co-activator complex of Oct-1 in the transcriptional induction of histone H2B gene during the S phase of the cell cycle. Intriguingly, GAPDH interacts directly with Oct-1 and it has an intrinsic activation domain that can relate with the general transcription machinery.

GAPDH may also act as a glucose sensor in the cells stimulating autophagic degradation. Indeed, during glucose starvation, the AMPK-dependent GAPDH phosphorylation is essential for SIRT1 activation and stimulation of autophagy. In these conditions, cytoplasmic GAPDH is phosphorylated by activated AMPK prompting GAPDH to redistribute into the nucleus. Inside the nucleus, GAPDH directly interacts with SIRT1, displacing SIRT1's repressor and increasing SIRT1 deacetylase activity. In general, the multiple activities of GAPDH are related to its translocation to the nucleus or to different subcellular compartments in addition to the cytosolic localization, where its main role in glycolysis is well characterized.

Nuclear GAPDH is involved in a variety of functions such as autophagy and cell death, DNA repair, protection of telomeres from rapid degradation. The accumulation into the nucleus of GAPDH promotes the decline of its glycolytic activity. During oxidative stress, when DNA is damaged, simultaneous nitrosylation and translocation of GAPDH to the nucleus takes place and it can either bind to poly(ADP-ribose) polymerase 1 (PARP1) or directly to the damaged DNA. Under these stress conditions, PARP1 is activated by damaged DNA and synthesizes poly(ADP-ribose) using NAD⁺. Moreover, GAPDH translocated to the nucleus binds and activates PARP1. Over-activation of PARP1 depletes intracellular NAD⁺, therefore the NAD⁺ binding site of GAPDH becomes free and the enzyme acquires the ability to bind DNA. If a single stranded DNA fragment contains a cleaved site, GAPDH forms a stable covalent adduct with this damage. Thus, the formation of an irreversible complex of GAPDH with DNA seems to be a suicidal event, which hampers DNA repair in the case of accumulation of several damages and can be a factor leading to cell death.

Moreover, GAPDH has shown an intrinsic role in neuronal apoptosis since the presence of GAPDH into the nucleus is involved in the initiation of one or more apoptotic cascades. There are various case studies demonstrating the role of GAPDH in several neuronal diseases as HD and PD, and an attractive hypothesis is that GAPDH binds to the mutated proteins associated with these diseases resulting in a translocation to the nucleus, where the presence of GAPDH participates in the initiation of apoptosis. Accordingly, an increase in nuclear GAPDH in postmortem PD brain associated with the degeneration-sensitive substantia nigra dopaminergic neurons has been reported. Moreover, GAPDH is recognized as a major component of amyloid plaques in Alzheimer's diseased brains and it has been also reported to interact with neurodegenerative disease-associated proteins including the amyloid-β protein precursor (AβPP). Non-native GAPDH isoforms were able to bind to soluble AP species, indicating a direct involvement of GAPDH in amyloid aggregation.

Cytosolic GAPDH is also involved in apoptosis in a way mainly regulated by post-translational modifications and protein-protein interaction. Indeed, GAPDH is phosphorylated by Akt2 at Thr237 in the proximity of the binding site of Siah1, preventing its bond with Siah1 and apoptosis. The formation of the complex GAPDH/Akt2 is a mechanism identified in ovarian cancer cells to favor tumor cell survival and to avoid apoptosis. Another way through which cytosolic GAPDH is involved in tumor survival is the escape from caspase-independent cell death (CICD). By stabilizing Akt to its activated and phosphorylized form, overexpressed GAPDH prevents FoxO nuclear internalization regulating Bcl-6, a Bcl-xL inhibitor with anti-apoptotic functions.

Furthermore, many studies have demonstrated a functional link between cytosolic GAPDH and microtubules dynamics, vesicular trafficking and membrane recruitment and fusion. GAPDH can interact with tubulin and actin in normal conditions and with stress fibers during stress, which regulate its glycolytic function promoting its inactivation. These roles in cellular trafficking are regulated by post-translational phosphorylation of the enzyme, allowing it to take part in early secretory pathway transport. Serine/threonine kinases, facilitated by Rab2, act as regulators of GAPDH-mediated secretory activity, driving the direction of membrane transport. GAPDH also has a role as chaperone with the cellular labile heme. GAPDH helps in the transport and delivery of significant pool of cytosolic heme. It binds exogenous and endogenous heme, making it available to downstream protein targets that can be cytosolic (e.g., iNOS) or nuclear. In this way, GAPDH not only protects cells from heme toxicity but also involved in its mobilization.

In basal conditions, the level of GAPDH in mitochondria is very low and it strongly increases during stress conditions, such as serum deprivation and DNA damage. When GAPDH is expressed endogenously, mitochondrial GAPDH induces pro-apoptotic mitochondrial membrane permeabilization (MMP) via association with voltage dependent anion channel 1 (VDAC1).

Exogenous expression of mitochondria also causes loss of the inner transmembrane potential, matrix swelling, permeabilization of the inner-mitochondrial membrane, and the release of two pro-apoptotic proteins such as cytochrome c and apoptosis-inducing factor (AIF). Furthermore, during cardiac ischemia and reperfusion (I/R), GAPDH is found to be significantly associated with mitochondria, promoting direct uptake of damaged mitochondria into multi-organelle lysosomal-like (LL) structures for elimination, independently of the macroautophagy pathway.

The complex functions of this enzyme are tied to its translocation to different subcellular compartments. GAPDH-mediated autophagy and GAPDH aggregation may influence cancer cell growth and neurodegenerative disorders. Cancer-related factors can modulate GAPDH nuclear translocation, which is fundamental to regulate autophagy and cell death mechanisms. Autophagy stimulation by nuclear GAPDH may influence cancer cell fate acting as a prosurvival factor in cancer cells, supporting the energy consumption given by rapid cell proliferation even in stressing conditions. Moreover, the formation of aggregates of GAPDH or the interaction of GAPDH with specific disease-related proteins may be involved in neuronal cell death and mitochondrial dysfunction. Given its diverse and complex functionality, an effective therapeutic for safely and effectively modulating, inhibiting, and regulating the activity of GAPDH would provide a powerful tool in a broad range of medical fields.

Accordingly, there is a long-felt and unmet need for new compositions and methods to treat, inhibit, prevent or reduce disorders and diseases in a subject by administering one or more anti-GAPDH agents as well as to improve the performance, outcomes, and tolerability of existing therapeutic agents.

### SUMMARY OF THE INVENTION

In one aspect, the present disclosure includes a method of inhibiting GAPDH comprising administering to a subject in need of GAPDH-inhibition a compound.

In one aspect, the present disclosure includes method of inhibiting GAPDH in a subject in need thereof by administering a composition comprising a compound of the present disclosure to the subject.

In one aspect, the present disclosure includes a method of inhibiting about 30%, about 40%, about 50%, about 60%, about 70%, about 80%, about 90%, or about 100% of GAPDH activity in cells of a subject by administering a composition comprising a compound of the present disclosure to the subject.

In one aspect, the present disclosure includes a method of reducing or inhibiting production of adenosine triphosphate (ATP) in a subject in need thereof by administering a composition comprising a compound of the present disclosure to the subject.

In one aspect, the present disclosure includes a method of preventing, inhibiting or reducing at least one sign or symptom of a disease, disorder or condition caused by or associated with GAPDH activity in a subject in need thereof by administering a composition comprising a compound of the present disclosure to the subject.

In one aspect, the present disclosure includes a method of increasing production or localization of reactive species in a tumor of a subject in need thereof comprising administering a composition comprising a compound of the present disclosure to the subject.

In one aspect, the present disclosure includes a method of preventing, inhibiting or reducing at least one side effect of a drug administered to a subject suffering from a GAPDH-mediated disease, disorder, or condition, by administering a composition comprising a compound of the present disclosure to the subject.

In one aspect, the present disclosure includes a method of identifying inhibitors of GAPDH comprising combining a test compound with a solvent to form a solution, contacting the solution with recombinant GAPDH in buffer to form a reaction mixture, and subjecting aliquots of the reaction mixture to an enzyme activity assay, detecting change in NAD+ concentration in the enzyme activity assay, identifying a test compound that inhibit GAPDH by identifying a test compound that reduces NAD+ concentration in the enzyme activity assay compared to a control solvent.

In one aspect, the present disclosure includes a method of treating a subject suffering from a GAPDH-mediated disease, disorder, or condition comprising obtaining a biological sample comprising cells from a subject, lysing the cells, monitoring GAPDH activity in the lysed cells as a biomarker for GAPDH-mediated disease, and administering a composition comprising a GAPDH inhibitor to the subject.

In one aspect, the present disclosure includes a method for identifying a candidate suitable for treatment with a GAPDH-inhibitor compound comprising administering the GAPDH-inhibitor compound to a subject, obtaining peripheral blood mononuclear cells (PBMCs) from a subject, lysing the PBMCs, monitoring GAPDH activity in the lysed PBMCs, subjecting the lysed PBMCs to an enzyme activity assay, detecting changes in NAD+ concentration in the enzyme activity assay, monitoring inhibition of GAPDH by an administered GAPDH-inhibitor based on reduction of NAD+ concentration in the enzyme activity assay compared to a control solvent, determining the degree of inhibition of GAPDH in the PBMCs, and identifying the subject as a suitable candidate for treating with the GAPDH-inhibitor compound if the degree of inhibition of GAPDH by the GAPDH-inhibitor compound is greater than a predetermined threshold.

In one aspect, the present disclosure includes a method of treatment comprising identifying the candidate suitable for treatment with a GAPDH-inhibitor according to the method of claim 20 or claim 21, and treating the candidate with a compound of the present disclosure.

In one aspect, the present disclosure includes a method of treating macular degeneration in a subject in need thereof by administering a composition comprising a compound of the present disclosure to the subject.

In some aspects, the present disclosure may include taurolidine, or a pharmaceutically acceptable salt, hydrate, ester, or solvate thereof, and compositions comprising taurolidine, or a pharmaceutically acceptable salt, hydrate, ester, or solvate thereof and an excipient, buffer, or carrier.

In one aspect, the present disclosure includes a complex or conjugate of Glyceraldehyde 3-phosphate dehydrogenase (GAPDH) with a compound of the present disclosure.

Other features and characteristics of the subject matter of this disclosure, as well as the methods of operation, functions of related elements of structure and the combination of parts, and economies of manufacture, will become more apparent upon consideration of the following description and the appended claims, all of which form a part of this specification.

### DETAILED DESCRIPTION

While aspects of the subject matter of the present disclosure may be embodied in a variety of forms, the following description are merely intended to disclose some of these forms as specific examples of the subject matter encompassed by the present disclosure. Accordingly, the subject matter of this disclosure is not intended to be limited to the forms or aspects so described and illustrated.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific aspects of the invention, but their usage does not delimit the invention, except as outlined in the claims.

The terms “inhibiting,” “reducing,” or “prevention,” or any variation of these terms, when used in the claims and/or the specification includes any measurable decrease or complete inhibition to achieve a desired result.

Anti-GAPDH agents of the present disclosure may be administered to any subject in need of inhibiting of GAPDH activity. Such subjects may be at risk of suffering from or suffering from a variety of diseases, disorders and conditions. For example, such diseases, disorders and conditions may be characterized by impaired glycolysis, impaired protein degradation pathways, uncontrolled protein aggregation, aerobic glycolysis, mitochondrial dysfunction, increased glucose uptake or metabolism, neovascularization, autoimmune reactions, immune reactions, excessive angiogenesis, dysfunctional apoptosis of normal cells, and/or impaired autophagy. As used herein, the phrase “GAPDH-mediated disorder, disease or condition” encompasses any one or more disorder, disease or condition in a subject in need of inhibiting of GAPDH activity including but not limited to diseases, disorders and conditions which may be characterized by impaired glycolysis, impaired protein degradation pathways, uncontrolled protein aggregation, aerobic glycolysis, mitochondrial dysfunction, increased glucose uptake or metabolism, neovascularization, autoimmune reactions, immune reactions, excessive angiogenesis, dysfunctional apoptosis of normal cells, and/or impaired autophagy, and including, but not limited to any one or more disorder, disease or condition discussed herein.

The present disclosure provides methods and compositions to inhibit GAPDH targeting cells with aerobic glycolysis. In this type of metabolism, only a small fraction of the glucose flux is used for energy production and can be decreased by GAPDH inhibition. Aerobic glycolysis is found in nearly all types of tumor cells, but not in normal cells. Accordingly, the present disclosure provides methods and compositions having broad spectrum anti-GAPDH activity without general toxicity to normal cells. Further, the present disclosure provides methods and compositions for regulating cells operating with an aerobic glycolytic energy metabolism, e.g., activated endothelial cells and activated immune cells.

In some aspects, the present disclosure provides methods and compositions to irreversibly inhibit GAPDH. As such, the present disclosure provides a surprising and unexpected advantage over existing therapies, e.g., antibodies, which require continuous dosing and are minimally effective. The present disclosure provides a way to permanently inactivate GAPDH by irreversibly binding to its active site.

In some aspects, the present disclosure provides methods and compositions to regulate mitochondrial function and protein production to reduce, inhibit, prevent and/or eliminate cancer stem cells (CSCs). In some aspects, the present disclosure provides methods and compositions to increase reactive species, e.g., reactive oxygen species, in tumors and cancerous cells, thereby reducing cancer cell viability without affecting normal cells. In some aspects, the present disclosure provides methods and compositions to induce reversion of desmoplastic tissue surrounding cancer cells/tumors to normal extracellular matrix. In some aspects, the present disclosure provides methods and compositions to reduce, inhibit, prevent and/or ablate cytokines. In some aspects, the present disclosure provides methods and compositions for administration to subjects having therapies/conditions that give rise to cytokine release or increased levels of cytokines. In some aspects, the present disclosure provides methods and compositions for reducing, inhibiting, preventing and/or ablating cytokines without interfering with targeted cancer cell cytotoxicity in immune therapies including but not limited to T-cell engaging therapies, e.g., CAR-T and bispecific therapies.

In some aspects, the present disclosure also provides methods and compositions for treating, reducing, inhibiting, or preventing Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticarial, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS), Eosinophilic Granulomatosis (EGPA), Cicatricial pemphigoid, Cogan's syndrome, Cold agglutinin disease, Congenital heart block, Coxsackie myocarditis, CREST syndrome (limited scleroderma), Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, graft versus host disease (GVHD), Granulomatosis with Polyangiitis, Graves' disease, Guillain-Barre syndrome, Hashimoto's thyroiditis, Hemolytic anemia, Henoch-Schonlein purpura (HSP), Herpes gestationis, pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease, tumors, cancers including, but not limited to carcinoma, leukemia, lymphoma, melanoma, myeloma, sarcoma, a metastatic solid tumor, and mixed-type cancers, skin diseases (including, but are not limited to, psoriasis, telangiectasia, wound granularization, scleroderma, neovascularization as a consequence of infection (e.g., cat scratch disease, bacterial ulceration, etc.)), macular degeneration or age-related blindness, diabetic ulcers, chronic ulcers and wounds, stroke, traumatic brain injury, neovascularization of the retina, neovascularization of the cornea (such as that caused by trachoma, infections, inflammation, transplantations or trauma), diabetic retinopathy, diabetic retinal edema, diabetic macula edema, ischemic retinopathy, hypertensive retinopathy, occlusive retinopathy, retinopathy of prematurity, neovascularization subsequent to trauma, neovascularization subsequent to infection, neovascularization subsequent to transplantation, neovascularization subsequent to retinal detachment or retinal degeneration, neovascular glaucoma, anterior chamber and/or anterior chamber angle neovascularization, choroidal neovascularization (CNV), subretinal neovascularization, retrolental fibroplasias, ocular histoplasmosis syndrome, myopic degeneration, angioid streaks, uveitis, rubeosis, retrolental fibroplasias, ocular histoplasmosis, and idiopathic central serous chorioretinopathy, amyotrophic lateral sclerosis, sarcoidosis, scleroderma, lupus, Parkinson's disease, sclerosis, Stevens-Johnson syndrome, neoplasia, Von Willebrand disease, vasculitis, and Kawasaki disease.

The present disclosure also provides methods and compositions for treating subjects suffering from cardiovascular diseases including but not limited to atherosclerosis, restenosis, atheroma, and haemangioma. Atherosclerosis is a form of chronic vascular injury in which some of the normal vascular smooth cells (VSMC) in the artery wall change their nature and develop dense networks of capillaries in atherosclerotic plaques. These fragile microvessels can cause hemorrhages, leading to blood clotting, with a subsequent decreased blood flow to the heart muscle and heart attack. Restenosis typically occurs after coronary artery bypass surgery, endarterectomy, and heart transplantation, and particularly after heart balloon angioplasty, atherectomy, laser ablation or endovascular stenting.

As used herein, the terms “substantially” and “substantial” refer to a considerable degree or extent. When used in conjunction with, for example, an event, circumstance, characteristic, or property, the terms can refer to instances in which the event, circumstance, characteristic, or property occurs precisely as well as instances in which the event, circumstance, characteristic, or property occurs to a close approximation, such as accounting for typical tolerance levels or variability of the examples described herein.

As used herein, the term “about” is used to provide flexibility to a numerical range endpoint by providing that a given value may be “a little above” or “a little below” the endpoint. The degree of flexibility of this term can be dictated by the particular variable and would be within the knowledge of those skilled in the art to determine based on experience and the associated description herein. For example, in one aspect, the degree of flexibility can be within about ±10% of the numerical value. In another aspect, the degree of flexibility can be within about ±5% of the numerical value. In a further aspect, the degree of flexibility can be within about ±2%, ±1%, or ±0.05%, of the numerical value.

Generally herein, the term “or” includes “and” and “and/or.”

As used herein, a plurality of compounds or steps may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

The compounds of the invention may be useful in a free acid form, a free base form, in the form of pharmaceutically acceptable salts, pharmaceutically acceptable hydrates, pharmaceutically acceptable esters, pharmaceutically acceptable solvates, pharmaceutically acceptable prodrugs, pharmaceutically acceptable metabolites, and in the form of pharmaceutically acceptable stereoisomers. These forms are all within the scope of the invention. In practice, the use of these forms amounts to use of the neutral compound.

“Pharmaceutically acceptable salt”, “hydrate”, “ester” or “solvate” refers to a salt, hydrate, ester, or solvate of the inventive compounds which possesses the desired pharmacological activity and which is neither biologically nor otherwise undesirable. Organic acids can be used to produce salts, hydrates, esters, or solvates such as acetate, adipate, alginate, aspartate, benzoate, benzenesulfonate, p-toluenesulfonate, bisulfate, sulfamate, sulfate, naphthylate, butyrate, citrate, camphorate, camphorsulfonate, cyclopentanepropionate, digluconate, dodecylsulfate, ethanesulfonate, fumarate, glucoheptanoate, glycerophosphate, hemisulfate heptanoate, hexanoate, 2-hydroxyethanesulfonate, lactate, maleate, methanesulfonate, 2-naphthalenesulfonate, nicotinate, oxalate, tosylate and undecanoate. Inorganic acids can be used to produce salts, hydrates, esters, or solvates such as hydrochloride, hydrobromide, hydroiodide, and thiocyanate. Other pharmaceutically acceptable salts include, but are not limited to, hydrochloride, hydrobromide, sulphate, phosphate, tartrate, fumarate, maleate, oxalate, acetate, propionate, succinate, mandelate, mesylate, besylate and tosylate.

Salts, hydrates, esters, or solvates may also be formed with organic bases. Pharmaceutically acceptable base addition salts of acidic compounds may be formed with organic and inorganic bases by conventional methods. For example, alkali metal and alkaline earth metal hydroxides, carbonates and bicarbonates such as sodium hydroxide, potassium hydroxide, calcium hydroxide, potassium carbonate, sodium bicarbonate, magnesium carbonate and the like, ammonia, primary, secondary and tertiary amines and the like. Also aluminum salts of the instant compounds may be obtained by treating the corresponding sodium salt with an appropriate aluminum complex such as, for example, aluminum chloride hexahydrate, and the like. Non-toxic organic bases include, but are not limited to, triethylamine, butylamine, piperazine, and tri(hydroxymethyl)-methylamine. Examples of suitable base salts, hydrates, esters, or solvates include hydroxides, carbonates, and bicarbonates of ammonia, alkali metal salts such as sodium, lithium and potassium salts, alkaline earth metal salts such as calcium and magnesium salts, aluminum salts, and zinc salts. Organic bases suitable for the formation of pharmaceutically acceptable base addition salts, hydrates, esters, or solvates of the compounds of the present invention include those that are non-toxic and strong enough to form such salts, hydrates, esters, or solvates. For purposes of illustration, the class of such organic bases may include mono-, di-, and trialkylamines, such as methylamine, dimethylamine, triethylamine and dicyclohexylamine; mono-, di- or trihydroxyalkylamines, such as mono-, di-, and triethanolamine; amino acids, such as arginine and lysine; guanidine; N-methyl-glucosamine; N-methyl-glucamine; L-glutamine; N-methyl-piperazine; morpholine; ethylenediamine; N-benzyl-phenethylamine; (trihydroxy-methyl)aminoethane; and the like. See, for example, “Pharmaceutical Salts,” J. Pharm. Sci., 66:1, 1-19 (1977). Accordingly, basic nitrogen-containing groups can be quaternized with agents including: lower alkyl halides such as methyl, ethyl, propyl, and butyl chlorides, bromides and iodides; dialkyl sulfates such as dimethyl, diethyl, dibutyl and diamyl sulfates; long chain halides such as decyl, lauryl, myristyl and stearyl chlorides, bromides and iodides; and aralkyl halides such as benzyl and phenethyl bromides.

“Pharmaceutically acceptable prodrug” refers to a derivative of the inventive compounds which undergoes biotransformation prior to exhibiting its pharmacological effect(s). The prodrug is formulated with the objective(s) of improved chemical stability, improved patient acceptance and compliance, improved bioavailability, prolonged duration of action, improved organ selectivity, improved formulation (e.g., increased hydrosolubility), and/or decreased side effects (e.g., toxicity). The prodrug can be readily prepared from the inventive compounds using methods known in the art, such as those described by Burger's Medicinal Chemistry and Drug Chemistry, Fifth Ed., Vol. 1, pp. 172-178, 949-982 (1995). For example, the inventive compounds can be transformed into prodrugs by converting one or more of the hydroxy or carboxy groups into esters.

“Pharmaceutically acceptable metabolite” refers to drugs that have undergone a metabolic transformation. After entry into the body, most drugs are substrates for chemical reactions that may change their physical properties and biologic effects. These metabolic conversions, which usually affect the polarity of the compound, alter the way in which drugs are distributed in and excreted from the body. However, in some cases, metabolism of a drug is required for therapeutic effect. For example, anticancer drugs of the antimetabolite class must be converted to their active forms after they have been transported into a cancer cell. Since must drugs undergo metabolic transformation of some kind, the biochemical reactions that play a role in drug metabolism may be numerous and diverse. The main site of drug metabolism is the liver, although other tissues may also participate.

Furthermore, certain compositions, concentrations, dosage regimens, dosage amounts, syndromes or conditions, steps, or the like may be discussed in the context of one specific aspect. It is understood that this is merely for convenience, and such disclosure is equally applicable to other aspects found herein. For example, a list of method steps, active agents, kits or compositions described with respect to a method of administering an anti-GAPDH agent of the present disclosure would find direct support for aspects related to method steps, active agents, kits or compositions of, e.g., the following: treating, preventing, inhibiting or reducing at least one sign or symptom of a disease, disorder or condition caused by or associated with GAPDH activity; treating, preventing, inhibiting or reducing at least one side effect of a drug administered to a subject suffering from a disease, disorder or condition caused by or associated with GAPDH activity; treating, preventing, inhibiting or reducing the incidence of a sign or symptom of a disease, disorder or condition caused by or associated with GAPDH activity; modulating vascularization; regulating vascularization; modulating angiogenesis; and regulating GAPDH activity, even if those method steps, active agents, kits or compositions are not re-listed in the context of that aspect in the specification.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilizing (i.e. not worsening) the state of disease, delaying or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. In addition to being useful as methods of treatment, the methods described herein may be useful for the prevention or prophylaxis of disease. As used herein, the term “treating” may refer to any administration of a compound of the present invention and includes: (i) preventing or inhibiting the disease in a mammal, e.g., a human, that is experiencing or displaying the pathology or symptomatology of the diseased (i.e., arresting further development of the pathology and/or symptomatology); or (ii) ameliorating the disease in a mammal, e.g., a human that is experiencing or displaying the pathology or symptomatology of the disease (i.e., reversing the pathology and/or symptomatology). The term “controlling” includes preventing, treating, eradicating, ameliorating or otherwise reducing the severity of the condition being controlled.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0” should be interpreted to include not only the explicitly recited values of about 0.01 to about 2.0, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. Furthermore, such an interpretation should apply regardless of the breadth of the range or the characteristics being described. Additionally, it is noted that all percentages are in weight, unless specified otherwise.

In understanding the scope of the present disclosure, the terms “including” or “comprising” and their derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps. It is understood that reference to any one of these transition terms (i.e. “comprising,” “consisting,” or “consisting essentially”) provides direct support for replacement to any of the other transition term not specifically used. For example, amending a term from “comprising” to “consisting essentially of” would find direct support due to this definition.

The present disclosure relates to using an “N-methylol transfer agent” and “N-methylol donating compound,” collectively referred to as “N-methylol transfer agent” or “compounds of the present disclosure.” The terms “N-methylol transfer agent” and “N-methylol donating compound” and cognates thereof indicate a compound which contains or is capable of producing a methylol molecule under physiological conditions. N-methylol transfer agents include compounds such as Taurolidine and Taurultam, and their derivatives, including taurinamide and urea derivatives. The compounds Taurolidine and Taurultam are disclosed in U.S. Pat. No. 5,210,083, which is incorporated herein by reference, and are structurally depicted as follows:

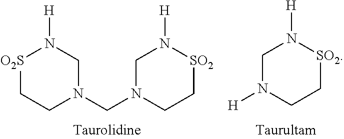

Certain N-methylol transfer agents are Taurolidine, Taurultam, and mixtures thereof. Other suitable N-methylol-containing compounds include taurinamide derivatives and urea derivatives, examples of which are identified herein and shown in the Figures. Examples of specific derivatives of Taurolidine, Taurultam, taurinamide and urea which may be useful in the present invention also can be found in WO 01/39763A2, the disclosures of which are hereby incorporated by reference.

A “derivative” of Taurolidine or Taurultam refers to a sulfonamide compound which possesses at least 10% of the neoplastic activity of Taurolidine or Taurultam, respectively. Some examples of such compounds include but are not limited to 1,3,-dimethylol-5,5-dimethylhydantoin, hexamethylene tetramine, or noxythiolin. Other N-methylol transfer agents contemplated for use with the invention include cyclotaurolidine or N-methyltaurinamide and compounds disclosed in U.S. Pat. No. 9,028,866, which is incorporated herein by reference in its entirety.

In certain aspects, the N-methylol transfer agent is Taurolidine, Taurultam, 1183B (cyclo-taurolidine or 7-oxa-2[λ]⁶-thia-1,5-diazabicyclo[ 3.3.1]nonane-2,2-dione), N-methylol taurinamide, 1,3,-dimethylol-5,5-dimethylhydantoin, hexamethylene tetramine, or noxythiolin, N-methyltaurinamide, a substance which forms N-methylol taurinamide, or any combination thereof. According to certain embodiments, the agent is Taurolidine, Taurultam, or a combination thereof.

In certain aspects, Taurolidine or Taurultam is used for inhibiting GAPDH and for treating, preventing, inhibiting or reducing at least one sign or symptom of a disease, disorder, condition, symptom caused by or associated with GAPDH activity in accordance with the disclosure herein, e.g., including but not limited to a disease, disorder, condition or symptom caused by or associated with impaired glycolysis, impaired protein degradation pathways, uncontrolled protein aggregation, aerobic glycolysis, mitochondrial dysfunction, increased glucose uptake or metabolism, neovascularization, autoimmune reactions, immune reactions, excessive angiogenesis, dysfunctional apoptosis of normal cells, and/or impaired autophagy.

In certain aspects, the present disclosure also includes GAPDH bound to one or more of the compounds of the present disclosure. For example, the present disclosure includes a complex or conjugate of GAPDH and one or more of the foregoing compounds of the present disclosure.

As used herein, a “complex” refers to one or more of the compounds of the present disclosure complexed with GAPDH, wherein at least one compound of the present disclosure is bound to or sequestered by GAPDH. As used herein, a “conjugate” refers to one or more of the compounds of the present disclosure covalently bound to GAPDH.

In some aspects, the one or more of the foregoing compounds may be covalently bound to one or more of the cysteines of GAPDH. In some aspects, the one or more of the foregoing compounds may be covalently bound to the catalytic (active site) cysteine-SH of GAPDH,

In certain aspects, the present disclosure includes a method of inhibiting NFkB (NF kappa B) by administering a compound of the present disclosure. In certain aspects, the present disclosure includes a method of decreasing expression of Bcl-2 by administering a compound of the present disclosure. In certain aspects, the present disclosure includes a method of increasing expression of Bax by administering a compound of the present disclosure.

In certain aspects, the invention also relates to compositions, e.g., pharmaceutical compositions, containing the compounds, complexes, or conjugates described herein, including pharmaceutically acceptable solutions thereof, as well as administrable compositions, kits, medical devices, and pharmaceutical containers containing the compositions of the present disclosure.

The terms “effective amount” or “therapeutically effective amount” described herein means the amount of the subject compound that will elicit the biological or medical response of a tissue, system, animal or human that is being sought by the researcher, veterinarian, medical doctor or other clinician. In one example, the therapeutically effective amount comprises about 0.0001 to about 10,000 mg/kg, about 0.001 mg/kg to about 5,000 mg/kg, about 0.01 mg/kg to about 1,000 mg/kg, about 0.05 mg/kg to about 750 mg/kg, about 0.1 mg/kg to about 600 mg/kg, about 1 mg/kg to about 500 mg/kg, about 10 mg/kg to about 400 mg/kg, about 20 mg/kg to about 300 mg/kg, about 200 mg/kg to about 500 mg/kg, about 300 mg/kg to about 400 mg/kg, about 250 mg/kg, 300 mg/kg, 400 mg/kg, 420 mg/kg, 450 mg/kg, about 500 mg/kg, or an dosage amount or range within any of the disclosed ranges of body weight of the subject.

The terms “administration of” or “administering a” compound as used herein should be understood to mean providing a compound of the invention to the individual in need of treatment in a form that can be introduced into that individual's body, e.g., intravenously, subcutaneously, intramuscularly, topically, orally, intraperitoneally, ophthalmically, by intravitreal injection, intrathecally, intranasally, intrapulmonary, transdermally, intraocularly, by inhalation, transtracheally, intravitreally, or a combination thereof. In some aspects, a compound of the invention may be administered in a therapeutically useful form and therapeutically useful amount, including, but not limited to: oral dosage forms, such as tablets, capsules, syrups, suspensions, and the like; injectable dosage forms, such as intravenous (IV), intramuscular (IM), or intraperitoneal (IP), intranasal, and the like; enteral or parenteral, transdermal dosage forms, including creams, jellies, powders, or patches; buccal dosage forms; inhalation powders, sprays, suspensions, and the like; and rectal suppositories.

Depending upon the particular route of administration desired a variety of pharmaceutically acceptable carriers well known in the art may be used. These include solid or liquid fillers, diluents, hydrotropes, surface-active agents, and encapsulating substances. Optional pharmaceutically active materials may be included, which do not substantially interfere with the activity of the one or more N-methylol transfer agents.

As used herein the term “intravenous administration” includes injection, infusion, and other modes of intravenous administration.

The term “pharmaceutically acceptable” as used herein to describe a carrier, diluent or excipient must be compatible with the other ingredients of the formulation and not deleterious to the recipient thereof.

In one aspect, the present disclosure includes administering one or more compounds of the present disclosure alone or in combination with at least one second active agent. For example, in some aspects, the present disclosure includes administering one or more compounds of the present disclosure with an anti-angiogenesis agent, anti-autoimmune agent, and/or anti-neoplastic agent to a subject in need thereof.

In one aspect, the present disclosure includes administering one or more compounds of the present disclosure to inhibit GAPDH activity in a subject in need thereof. In one aspect, the present disclosure includes a method of inhibiting about 30%, about 40%, about 50%, about 60%, about 70%, about 80%, about 90%, or about 100% of GAPDH activity in cells of a subject.

In one aspect, the present disclosure includes reducing or inhibiting production of adenosine triphosphate (ATP) in a subject in need thereof by administering one or more compounds of the present disclosure to inhibit GAPDH activity in the subject.

In one aspect, the present disclosure includes inhibiting GAPDH activity by administering one or more compounds of the present disclosure to a subject in need thereof to treat, prevent, inhibit or reduce at least one sign or symptom of a disease, disorder or condition caused by or associated with GAPDH activity, e.g., including but not limited to a disease, disorder or condition caused by or associated with impaired glycolysis, impaired protein degradation pathways, uncontrolled protein aggregation, aerobic glycolysis, mitochondrial dysfunction, increased glucose uptake or metabolism, neovascularization, autoimmune reactions, immune reactions, excessive angiogenesis, dysfunctional apoptosis of normal cells, and/or impaired autophagy.

In some aspects, the present disclosure includes inhibiting GAPDH activity by administering one or more compounds of the present disclosure to a subject in need thereof to irreversibly inhibit GAPDH. In some aspects, the present disclosure includes inhibiting GAPDH activity by administering one or more compounds of the present disclosure to a subject in need thereof to regulate mitochondrial function and protein production to reduce, inhibit, prevent and/or eliminate cancer stem cells (CSCs). In some aspects, the present disclosure includes inhibiting GAPDH activity by administering one or more compounds of the present disclosure to a subject in need thereof to increase production or localization of reactive species, e.g., reactive oxygen species, in tumors and cancerous cells, thereby reducing cancer cell viability without affecting normal cells. In some aspects, the present disclosure includes inhibiting GAPDH activity by administering one or more compounds of the present disclosure to a subject in need thereof to induce reversion of desmoplastic tissue surrounding cancer cells/tumors to normal extracellular matrix. In some aspects, the present disclosure includes inhibiting GAPDH activity by administering one or more compounds of the present disclosure to reduce, inhibit, prevent and/or ablate cytokines. In some aspects, the present disclosure includes treating a subject having therapies/conditions that give rise to cytokine release or increased levels of cytokines by co-administering one or more compounds of the present disclosure to the subject to prevent, inhibit, or reduce cytokine release or increased levels of cytokines in the subject. In some aspects, the present disclosure includes inhibiting GAPDH activity by administering one or more compounds of the present disclosure to a subject in need thereof to reduce, inhibit, prevent and/or ablate cytokines without interfering with targeted cancer cell cytotoxicity in immune therapies including but not limited to T-cell engaging therapies, e.g., CAR-T and bispecific therapies.

In some aspects, the present disclosure includes methods and compositions for treating a subject having cancer, autoimmune disease, angiogenesis or other disease, disorder, condition or symptom disclosed herein, comprising selecting a subject having cancer, autoimmune disease, angiogenesis or other disease, disorder, condition or symptom disclosed herein, associated with GAPDH and administering to the selected subject one or more GAPDH inhibitors comprising taurolidine compounds of the present disclosure.

In some aspects, the present disclosure includes methods and compositions for treating cancer, autoimmune disease, neovascularization, and/or excessive angiogenesis associated with GAPDH in a subject comprising administering to the subject one of more GAPDH inhibitors comprising taurolidine compounds of the present disclosure.

In some aspects, the present disclosure includes methods for selecting a subject having cancer, autoimmune disease, neovascularization, and/or excessive angiogenesis associated with GAPDH for treatment with one or more taurolidine compounds comprising detecting GAPDH in a biological sample of the subject and selecting the subject for treatment with one of more taurolidine compounds of the present disclosure. In some aspects, the cancer, autoimmune disease, neovascularization, and/or excessive angiogenesis associated with GAPDH in a subject is determined by isolating a sample of cells or biological sample from a subject and assessing GAPDH activity in the cells or biological sample.

In some aspects, the present disclosure includes methods for screening for GAPDH inhibition using N-methylol transfer agents including Taurolidine, Taurultam, urea derivatives, and mixtures thereof by contacting a cell or biological sample containing GAPDH with N-methylol transfer agents including Taurolidine, Taurultam, urea derivatives, and mixtures thereof and determining whether GAPDH is inhibited in the cell or biological sample and selecting from the one or more N-methylol transfer agents at least one compound that inhibits GAPDH. In some aspects, a GAPDH inhibition of above a threshold (e.g., by at least 30% over control) indicates that the compound has anticancer, anti-autoimmune, anti-neovascularization, and/or anti-excessive angiogenesis activity.

In some aspects, the present disclosure includes methods for determining if GAPDH is inhibited by one or more N-methylol transfer agents by contacting a cell or biological sample containing GAPDH with one or more N-methylol transfer agents and determining whether GAPDH is inhibited in the cell or biological sample.

In some aspects, the present disclosure includes methods of evaluating anticancer, autoimmune, neovascularization, and/or excessive angiogenesis properties of N-methylol transfer agents for treating cancer, autoimmune disease, neovascularization, and/or excessive angiogenesis comprising contacting a cell or biological sample with N-methylol transfer agents and determining whether GAPDH is inhibited in the cell or biological sample, wherein GAPDH inhibition by a N-methylol transfer agent indicates that the N-methylol transfer agent is useful for treating cancer, autoimmune diseases, neovascularization, and/or excessive angiogenesis.

Anti-GAPDH agents of the present disclosure may be administered to subjects at risk of suffering from or suffering from a variety of diseases, disorders and conditions. Such diseases, disorders and conditions may be characterized by neovascularization and/or excessive angiogenesis. The present disclosure also provides methods and compositions for modulating and regulating vascularization, modulating and regulating angiogenesis, and preventing, treating, inhibiting, or reducing neovascularization and/or excessive angiogenesis also referred to as angiogenesis-associated or neovascularization-associated diseases, disorders and conditions. Non-limiting examples of such diseases, disorders and conditions include one or more of tumors, cancers including, but not limited to carcinoma, leukemia, lymphoma, melanoma, myeloma, sarcoma, a metastatic solid tumor, and mixed-type cancers, skin diseases (including, but are not limited to, psoriasis, telangiectasia, wound granularization, scleroderma, neovascularization as a consequence of infection (e.g., cat scratch disease, bacterial ulceration, etc.)), macular degeneration or age-related blindness, diabetic ulcers, chronic ulcers and wounds, stroke, traumatic brain injury, neovascularization of the retina, neovascularization of the cornea (such as that caused by trachoma, infections, inflammation, transplantations or trauma), diabetic retinopathy, diabetic retinal edema, diabetic macula edema, ischemic retinopathy, hypertensive retinopathy, occlusive retinopathy, retinopathy of prematurity, neovascularization subsequent to trauma, neovascularization subsequent to infection, neovascularization subsequent to transplantation, neovascularization subsequent to retinal detachment or retinal degeneration, neovascular glaucoma, anterior chamber and/or anterior chamber angle neovascularization, choroidal neovascularization (CNV), subretinal neovascularization, retrolental fibroplasias, ocular histoplasmosis syndrome, myopic degeneration, angioid streaks, uveitis, rubeosis, retrolental fibroplasias, ocular histoplasmosis, and idiopathic central serous chorioretinopathy, amyotrophic lateral sclerosis, sarcoidosis, scleroderma, lupus, Parkinson's disease, sclerosis, Stevens-Johnson syndrome, neoplasia, Von Willebrand disease, vasculitis, and Kawasaki disease.

The present disclosure also provides methods and compositions for treating subjects suffering from cardiovascular diseases including but not limited to atherosclerosis, restenosis, atheroma, and haemangioma Atherosclerosis is a form of chronic vascular injury in which some of the normal vascular smooth cells (VSMC) in the artery wall change their nature and develop dense networks of capillaries in atherosclerotic plaques. These fragile microvessels can cause hemorrhages, leading to blood clotting, with a subsequent decreased blood flow to the heart muscle and heart attack. Restenosis typically occurs after coronary artery bypass surgery, endarterectomy, and heart transplantation, and particularly after heart balloon angioplasty, atherectomy, laser ablation or endovascular stenting. It involves extensive growth of microvessels. By inhibiting angiogenesis in the cardiovascular tissue, the methods provided herein are useful for treating these cardiovascular diseases.

In one aspect, the present disclosure relates to treating macular degeneration. In particular, an ophthalmic formulation containing the compounds of the present disclosure are administered to a subject in need thereof. Ophthalmic indications according to the present disclosure include all forms of diabetic retinopathy in people with or without diabetic macular edema and specifically diabetic macular edema. Diabetic retinopathy is a serious condition that affects millions of people. In one aspect, compositions of the present disclosure are administered by intravitreal injection.

In some aspects, the present disclosure includes inhibiting GAPDH activity by administering one or more compounds of the present disclosure to a subject in need of reduce, inhibit, and/or prevent neovascularization and/or excessive angiogenesis in a subject. In some aspects, the at least one sign or symptom may include rash, muscle pain, joint pain, fatigue, anemia, inflammation, abdominal pain, abdominal bloating, diarrhea, nausea, acid reflux, weight gain, fever, ongoing headaches, bleeding complications (e.g., hemorrhage), hypertension, hypotension, low blood counts, tumor-growth, cachexia, light sensitivity, eye redness, eye irritation, or a combination thereof.

In one aspect, the present disclosure includes preventing, inhibiting or reducing at least one side effect of a drug administered to a subject suffering from disease, disorder or condition caused by or associated with neovascularization and/or excessive angiogenesis by inhibiting GAPDH activity by co-administering one or more N-methylol transfer agents to the subject. In some aspects, the at least one side effect may include one or more of bleeding complications (e.g., hemorrhage), hypertension, diarrhea, fatigue, low blood counts, reduced wound healing, itchy, dry or flaky skin, dry or watery eyes, pain, headaches, rash, dizziness, weight loss, hair loss, swelling, unusual bruising, seizure, muscle weakness, numbness, infection, fever, chills, aches, pain, poor appetite, change in weight, joint pain/swelling, or a combination thereof.

In one aspect, the present disclosure includes methods and compositions for increasing the therapeutic index of a chemotherapeutic drug by (e.g., lowering toxicity, increasing tumor uptake of the drug, increasing efficacy, etc.) inhibiting GAPDH activity by co-administering one or more N-methylol transfer agents of the present disclosure with the chemotherapeutic drug. In some aspects, the chemotherapeutic drug may include trastuzumab, alemtuzumab, bevacizumab, blinatumomab, brentuximab vedotin, infliximab, eculizumab, certolizumab, daclizumab, cetuximab, denosumab, dinutuximab, ibritumomab tiuxetan, ipilimumab, nivolumab, obinutuzumab, ofatumumab, panitumumab, pembrolizumab, pertuzumab, rituximab, trastuzumab. In some aspects, the combination increases the therapeutic index by rendering the co-therapy less toxic. The lower toxicity allows more chemotherapeutic drug(s) to be delivered while maintaining acceptable side effects. It is also contemplated that the co-therapy is more efficacious and, as such, less chemotherapeutic drug can be used to get the same results provided by previous compositions.

The phrases “co-administering” or “administering in combination” as used herein mean that two (or more) agents are administered in temporal juxtaposition. The co-administration or combination may be effected by the two agents being mixed into a single formulation, or by the two agents being administered separately but simultaneously, or separately and within a short time of each other. For example, in general the two agents are co-administered within the time range of 6-168 hours. In this case, the agents may be administered in either order, i.e. the chemotherapeutic drug may be administered first, or the one or more N-methylol transfer agents of the present disclosure may be administered first. In some aspects, the two agents are co-administered in a single formulation, or are co-administered sequentially and separately.

In one aspect, this disclosure relates to a method of reducing chemotherapy drug-related toxicity in a patient treated with a chemotherapy drug and at risk of such toxicity, which method comprises treating said patient with one or more N-methylol transfer agents and a chemotherapy drug, such that said patient has reduced risk of chemotherapy drug-related toxicity. In one embodiment, the chemotherapy drug-related toxicity is cardiotoxicity, nephrotoxicity, hepatotoxicity, pulmonary toxicity, dermatologic toxicity, or gastrointestinal toxicity. For example, some chemotherapeutic drugs may cause direct injury to the heart (either acute or chronic), including anthracyclines. Chemotherapy drugs, including cisplatin, cyclophosphamide, and ifosfamide, produce urinary tract/kidney toxicity. Drugs with pulmonary toxicity, including bleomycin, can cause severe pulmonary effects. Dermatologic toxicity is also common with chemotherapeutic drugs, and include transient rash (carmustine, cytarabine, gemcitabine, asparaginase, and procarbazine), photosensitivity (Mitomycin, 5-FU, methotrexate, vinblastine, and dacarbazine), dermatitis, hyperpigmentation, urticaria, nail changes, alopecia, and radiation recall. Gastrointestinal toxicity, including stomatitis or diarrhea, is also common.

In some aspects, the patient suffers from cancers or tumors including, but not limited to biliary tract cancer; brain cancer, including glioblastomas and medulloblastomas; breast cancer; triple negative breast cancer; uterine cancer; tubal cancer; cervical cancer; choriocarcinoma; colon cancer; bladder cancer; endometrial cancer; retinoblastoma; vaginal cancer; vulvar cancer; esophageal cancer; mouth cancer; gastric cancer; kidney cancer; hematological neoplasms, including acute lymphocytic and myelogenous leukemia; multiple myeloma; AIDS-associated leukemias and adult T-cell leukemia lymphoma; intraepithelial neoplasms, including Bowen's disease and Paget's disease; liver cancer (hepatocarcinoma); lung cancer; head or neck cancers or oral cancers (mouth, throat, esophageal, nasopharyngeal, jaw, tonsil, nasal, lip, salivary gland, tongue, etc.); lymphomas, including Hodgkin's disease and lymphocytic lymphomas; neuroblastomas; neuroendocrine tumors; oral cancer, including squamous cell carcinoma; adrenal cancer; anal cancer; angiosarcoma; appendix cancer; bile duct cancer; bone cancer; carcinoid tumors; soft tissue sarcoma; rhabdomyosarcoma; eye cancer; ovarian cancer, including those arising from epithelial cells, stromal cells, germ cells and mesenchymal cells, and fallopian tube cancer; gallbladder cancer; pancreatic cancer; prostate cancer; rectal cancer; sarcomas, including leiomyosarcoma, rhabdomyosarcoma, liposarcoma, fibrosarcoma and osteosarcoma; skin cancer, including melanoma, Kaposi's sarcoma, basocellular cancer and squamous cell cancer; testicular cancer, including germinal tumors (seminoma, non-seminoma[teratomas, choriocarcinomas]), stromal tumors and germ cell tumors; penile cancer; hemangioendothelioma; gastrointestinal cancer; ureteral cancer; urethral cancer; spinal cancer; pituitary gland cancer; primary central nervous system (CNS) lymphoma; thyroid cancer, including thyroid adenocarcinoma and medullar carcinoma; and renal cancer including adenocarcinoma and Wilms tumor. In some aspects, cancers or tumors include breast cancer, prostate cancer, colorectal cancer, lymphoma, multiple myeloma, and melanoma.

Toxicity and therapeutic efficacy of such molecules can be determined by standard pharmaceutical procedures in cell cultures or experimental animals, e.g., by determining the LD50 (the dose lethal to 50% of the population) and the ED50 (the dose therapeutically effective in 50% of the population). The dose ratio of toxic to therapeutic effects is the therapeutic index, which can be expressed as the ratio LD₅₀/ED₅₀.

As used herein, the term “therapeutic index” with regard to a chemotherapeutic drug indicates safety of the chemotherapeutic drug. In some aspects, the therapeutic index can include a comparison of the amount of a therapeutic agent that causes the therapeutic effect (e.g., killing cancer cells) to the amount of the therapeutic agent that causes toxicity (e.g., liver toxicity). It is contemplated that according to certain embodiments an improved therapeutic index can occur using the compositions and/or methods described herein, including without limitation when: (1) the dosage of chemotherapeutic drug is increased above the current therapeutic dosages; (2) the dosage of chemotherapeutic drug remains the same as the current therapeutic dosages; or (3) the dosage of chemotherapeutic drug is decreased below the current therapeutic dosages. In some embodiments, the compositions and methods, including the scenarios in this paragraph can elicit improved or similar therapeutic effect as seen with the current therapeutic dosages with no worse, fewer, or no toxicities.

In one aspect, the present disclosure includes a method of inhibiting GAPDH activity by administering one or more compounds of the present disclosure to a subject in need thereof to down-regulate vascularization by administering one or N-methylol transfer agents to a subject, thereby preventing neovascularization in the subject.

In one aspect, the present disclosure includes a method of inhibiting GAPDH activity by administering one or more compounds of the present disclosure to a subject in need thereof to down-regulate angiogenesis by administering one or more N-methylol transfer agents to a subject, thereby preventing undesired excessive angiogenesis in the subject.

In one aspect, the present disclosure includes a method of inhibiting GAPDH activity by administering one or more compounds of the present disclosure to a subject in need thereof to inhibit impaired glycolysis by administering one or more N-methylol transfer agents to a subject, thereby preventing undesired excessive angiogenesis in the subject.

In one aspect, the present disclosure includes a method of inhibiting GAPDH activity by administering one or more compounds of the present disclosure to a subject in need thereof to prevent, inhibit, reduce, or reverse impaired protein degradation pathways by administering one or more N-methylol transfer agents to a subject, thereby preventing undesired excessive angiogenesis in the subject.

In one aspect, the present disclosure includes a method of inhibiting GAPDH activity by administering one or more compounds of the present disclosure to a subject in need thereof to uncontrolled protein aggregation by administering one or more N-methylol transfer agents to a subject, thereby preventing undesired excessive angiogenesis in the subject.

In one aspect, the present disclosure includes a method of inhibiting GAPDH activity by administering one or more compounds of the present disclosure to a subject in need thereof to aerobic glycolysis by administering one or more N-methylol transfer agents to a subject, thereby preventing undesired excessive angiogenesis in the subject.

In one aspect, the present disclosure includes a method of inhibiting GAPDH activity by administering one or more compounds of the present disclosure to a subject in need thereof to mitochondrial dysfunction,

In one aspect, the present disclosure includes a method of inhibiting GAPDH activity by administering one or more compounds of the present disclosure to a subject in need thereof to increased glucose uptake or metabolism by administering one or more N-methylol transfer agents to a subject, thereby preventing undesired excessive angiogenesis in the subject.

In one aspect, the present disclosure includes a method of inhibiting GAPDH activity by administering one or more compounds of the present disclosure to a subject in need thereof to autoimmune reactions by administering one or more N-methylol transfer agents to a subject, thereby preventing undesired excessive angiogenesis in the subject.

In one aspect, the present disclosure includes a method of inhibiting GAPDH activity by administering one or more compounds of the present disclosure to a subject in need thereof to immune reactions by administering one or more N-methylol transfer agents to a subject, thereby preventing undesired excessive angiogenesis in the subject.

In one aspect, the present disclosure includes a method of inhibiting GAPDH activity by administering one or more compounds of the present disclosure to a subject in need thereof to dysfunctional apoptosis of normal cells by administering one or more N-methylol transfer agents to a subject, thereby preventing undesired excessive angiogenesis in the subject.

In one aspect, the present disclosure includes a method of inhibiting GAPDH activity by administering one or more compounds of the present disclosure to a subject in need thereof to impaired autophagy by administering one or more N-methylol transfer agents to a subject, thereby preventing undesired excessive angiogenesis in the subject.

In one aspect, the present disclosure includes inhibiting, reducing or preventing GAPDH activity by administering one or more N-methylol transfer agents to a subject, wherein the one or more N-methylol transfer agents interact with the reactive (catalytic) cysteine-SH in the active center of the subject's GAPDH and thereby inactivate GAPDH in the subject.

In some aspects, the present disclosure includes decreasing catalytic activity of GAPDH in the subject in a dose- and time-dependent manner. For example, inhibition of GAPDH by the compounds of the present disclosure may be due to the inactivation of the enzyme, e.g., by covalent interaction with the catalytic cysteine of GAPDH. This interaction has a major impact on the pharmacokinetics and the dosing schedule of the compounds of the present disclosure in patients. In some aspects, once covalently inactivated, GAPDH activity can be restored only by the synthesis of new enzyme protein. The duration of the target inhibition is therefore determined by the half-life of the GAPDH enzyme. Measuring the blood level of the free compounds of the present disclosure, which are metabolized and excreted, becomes obsolete as indicator for the inhibition of the target. In some aspects, blood levels of the compounds of the present disclosure administered to a patient do not reflect the activity status of the enzyme due to this phenomenon. The duration of the inhibition of the enzyme will exceed by far the presence of the free the compounds of the present disclosure in the blood. Thus, the dosing intervals of the compounds of the present disclosure are based on the half-life of the GAPDH enzyme protein.

In one aspect, the patient is treated with one or more N-methylol transfer agents, or a combination thereof, administered intravenously, orally or a combination thereof. In one aspect, the patient is treated with taurolidine administered intravenously, orally or a combination thereof.

In one aspect, the patient is administered one or more N-methylol transfer agents or a combination thereof in conjunction with administration of one or more therapeutic drugs for treating subjects with a disease, disorder or condition caused by or associated with impaired glycolysis, impaired protein degradation pathways, uncontrolled protein aggregation, aerobic glycolysis, mitochondrial dysfunction, increased glucose uptake or metabolism, neovascularization, autoimmune reactions, immune reactions, excessive angiogenesis, dysfunctional apoptosis of normal cells, and/or impaired autophagy, e.g., anti-VEGF antibodies, bevacizumab, ranibizumab, brolucizumab, lapatinib, sunitinib, sorafenib, axitinib, cabozantinib, lenvatinib, ponatinib, ramucirumab, reorafenib, vandetanib, pazopanib, pegaptanib, bevasriranib, aflibercept, thiazolidinediones, conbercept, and lampalizumab, corticosteroids, immunosuppressants, e.g., cyclosporine, tacrolimus, anti-inflammatory drugs, e.g., dimethyl fumarate, sphingosine-1-phosphate (S1P) receptor modulators, e.g., siponimod, fingolimod, ceralifimod, ozanimod, ponesimod, autoimmune modulator peptides, e.g., glatiramer acetate and similar random-sized peptides, biologic drugs, e.g., antibodies, fusion proteins, and interferon-based drugs.

In some aspects, the present disclosure includes administering one or more N-methylol transfer agents in combination with one or more of tocilizumab, antihistamines, antipyretics, anti-inflammatory compounds, corticosteroids, glucocorticoids, TNF-inhibitors (e.g., etanercept), siltuximab, T cell-depleting antibody therapies such as alemtuzumab and antithymocyte globulins (ATG), IL-1R-based inhibitors (anakinra), ibrutinib and cyclophosphamide.

Compounds according to the invention can be administered by any suitable method. Solid dosage forms for oral administration include capsules, tablets, pills, powders, orally-disintegrating tablets, and granules. In such solid dosage forms, the provided composition is mixed with at least one inert, pharmaceutically acceptable excipient and/or fillers or extenders (e.g., starches, lactose, sucrose, glucose, mannitol, and silicic acid), binders (e.g., carboxymethylcellulose, alginates, gelatin, polyvinylpyrrolidinone, sucrose, and acacia), humectants (e.g., glycerol), disintegrating agents (e.g., agar, calcium carbonate, potato starch, tapioca starch, alginic acid, certain silicates, and sodium carbonate), solution retarding agents (e.g., paraffin), absorption accelerators (e.g., quaternary ammonium compounds), wetting agents (e.g., cetyl alcohol and glycerol monostearate), absorbents (e.g., kaolin and bentonite clay), and lubricants (e.g., talc, calcium stearate, magnesium stearate, solid polyethylene glycols, sodium lauryl sulfate), and mixtures thereof. In the case of capsules, tablets and pills, the dosage form may comprise buffering agents.

Solid compositions of a similar type may be employed as fillers in soft and/or hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the provided composition(s) only in, or targeting, a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

In certain aspects, capsules may contain an excipient formulation containing one or more of hydroxypropyl methylcellulose (HPMC), gelatin, meglumine, and fish gelatin. In certain aspects, a capsule may contain taurolidine and/or taurultam. The capsule may optionally further contain one or more of lycopene, ellagic acid (polyphenol), curcumin, piperine, delphinidin, resveratrol, isothiocyanates such as sulforaphane, capsaicin, and piperlongumine.

In certain aspects, this disclosure relates to administering an N-methylol transfer agents orally to a patient. In some aspects, an N-methylol transfer agent is formulated in capsules or tablets. In certain aspects, oral dosage forms contain between about 50-1000 mg of an N-methylol transfer agents. In certain aspects, oral dosage forms contain between about 100-500 mg of an N-methylol transfer agent. In certain aspects, oral dosage forms contain between about 200-400 mg of an N-methylol transfer agent. In certain aspects, oral dosage forms contain between about 250-350 mg of an N-methylol transfer agent. In certain aspects, the N-methylol transfer agent is taurolidine.

In some aspects, the N-methylol transfer agent is provided in a composition at a concentration of about 0.01 to about 500 μg/ml. In some aspects, the N-methylol transfer agentis provided in a composition at a concentration of about 0.1 to about 100 μg/ml. In some aspects, the N-methylol transfer agentis provided in a composition at a concentration of about 10 to about 50 μg/ml.

In some aspects, the N-methylol transfer agent is provided in a composition at a concentration of about 0.001 to about 5 wt. %, about 0.01 to about 3.5 wt. %, about 0.1 to about 3 wt. %, about 0.5 to about 2.5 wt. %, or about 1 to about 2 wt. % . In some aspects, the N-methylol transfer agentis provided in a composition at a concentration of about 0.01 to about 1.5%. In some aspects, the N-methylol transfer agentis provided in a composition at a concentration of about 0.1% to about 1%. In some aspects, the N-methylol transfer agentis provided in a composition at a concentration of about 100 to about 5000 μM, about 250 to about 2500 μM, about 500 to about 2000 μM, about 750 to about 1500 μM, about 1000 to about 1250 μM, or any other concentration within the recited ranges.

In some aspects, the N-methylol transfer agent is provided in a composition in a unit dosage form. As used herein, a “unit dosage form” is a composition containing an amount of N-methylol transfer agentthat is suitable for administration to an animal, such as a mammal, e.g., a human subject, in a single dose, according to a good medical practice. These compositions may contain from about 0.1 mg (milligrams) to about 500 mg, for example from about 5 mg to about 350 mg of N-methylol transfer agent. The frequency of treatment with the composition of the invention may be changed to achieve and maintain the desired target plasma level. Thus, non-limiting examples of treatment schedules include daily, twice daily, three times daily, weekly, biweekly, monthly, and combinations thereof. Alternatively, the composition of the invention may also be administered as a continuous infusion or a bolus following by one, two, three or more different continuous infusions, e.g., at different rates and dosages of administered drug, such regimens optionally interrupted by one or more additional bolus injections.

In certain aspects, one or more N-methylol transfer agents of the present disclosure are administered to a subject prior to administration of a therapeutic that is expected to lead to impaired glycolysis, impaired protein degradation pathways, uncontrolled protein aggregation, aerobic glycolysis, mitochondrial dysfunction, increased glucose uptake or metabolism, neovascularization, autoimmune reactions, immune reactions, excessive angiogenesis, dysfunctional apoptosis of normal cells, and/or impaired autophagy in the subject. For example, in one aspect, the one or more N-methylol transfer agents of the present disclosure are administered about 12 to 96, e.g., 24, 48 or 72, hours prior to administration of a therapeutic that is expected to lead to (e.g., cause or promote, either directly or indirectly) impaired glycolysis, impaired protein degradation pathways, uncontrolled protein aggregation, aerobic glycolysis, mitochondrial dysfunction, increased glucose uptake or metabolism, neovascularization, autoimmune reactions, immune reactions, excessive angiogenesis, dysfunctional apoptosis of normal cells, and/or impaired autophagy in the subject. In one aspect, the one or more N-methylol transfer agents of the present disclosure are administered in one or multiple doses prior to administration of a therapeutic that is expected to lead to impaired glycolysis, impaired protein degradation pathways, uncontrolled protein aggregation, aerobic glycolysis, mitochondrial dysfunction, increased glucose uptake or metabolism, neovascularization, autoimmune reactions, immune reactions, excessive angiogenesis, dysfunctional apoptosis of normal cells, and/or impaired autophagy in the subject. In certain aspects, one or more N-methylol transfer agents of the present disclosure are administered to a subject concurrently with a therapeutic that is expected to lead to impaired glycolysis, impaired protein degradation pathways, uncontrolled protein aggregation, aerobic glycolysis, mitochondrial dysfunction, increased glucose uptake or metabolism, neovascularization, autoimmune reactions, immune reactions, excessive angiogenesis, dysfunctional apoptosis of normal cells, and/or impaired autophagy in the subject. In certain aspects, the N-methylol transfer agentis administered to the subject within about 1 to about 24 hours, about 4 to about 18 hours, about 6 to about 15 hours, or about 8 to about 12 hours after administration to the subject of a therapeutic that is expected to promote impaired glycolysis, impaired protein degradation pathways, uncontrolled protein aggregation, aerobic glycolysis, mitochondrial dysfunction, increased glucose uptake or metabolism, neovascularization, autoimmune reactions, immune reactions, excessive angiogenesis, dysfunctional apoptosis of normal cells, and/or impaired autophagy in the subject.

In certain aspects, one or more N-methylol transfer agents of the present disclosure are administered according to a regimen during a period when impaired glycolysis, impaired protein degradation pathways, uncontrolled protein aggregation, aerobic glycolysis, mitochondrial dysfunction, increased glucose uptake or metabolism, neovascularization, autoimmune reactions, immune reactions, excessive angiogenesis, dysfunctional apoptosis of normal cells, and/or impaired autophagy angiogenesis is expected to occur. For example, in one aspect, the one or more N-methylol transfer agents of the present disclosure are administered daily, every other day, biweekly, or weekly for the patient's lifetime, until remission, multiple years, multiple months, a 2 to 12 week period, a 3 to 10 week period, or a 4 to 8 week period, before, during, and/or after administration a therapeutic that is expected to lead to impaired glycolysis, impaired protein degradation pathways, uncontrolled protein aggregation, aerobic glycolysis, mitochondrial dysfunction, increased glucose uptake or metabolism, neovascularization, autoimmune reactions, immune reactions, excessive angiogenesis, dysfunctional apoptosis of normal cells, and/or impaired autophagy in the subject.

In one aspect, the one or more N-methylol transfer agents are provided in a composition and is administered to a subject in need thereof at a total daily dosage may be about 0.001 g to about 1000 g, e.g., about 0.01 g to about 500 g, 0.1 to 300 g, 0.5 to 200 g, 1 g to 100 g, or any amount within the recited range. The daily dosage may be administered in the form of an orally administrable composition. The daily dosage may be administered in the form of a capsule, a tablet, or a pharmaceutically acceptable solution. The daily dosage may be administered in a form that contains a compound of the present disclosure at a concentration of about 0.01 to about 5% w/v, about 0.1 to about 3% w/v, about 0.5 to about 2.5% w/v, or about 1 to about 2% w/v.

The daily dosage may be administered in a form that contains one or more N-methylol transfer agents at a concentration of about 0.001 μg/ml to about 1000 μg/ml, about 0.01 μg/ml to about 750 μg/ml, about 0.05 μg/ml to about 500 μg/ml, about 0.1 μg/ml to about 300 μg/ml, about 0.5 μg/ml to about 200 μg/ml, about 1 μg/ml to about 100 μg/ml, about 5 μg/ml to about 50 μg/ml, about 10 μg/ml to about 25 μg/ml, or about 15 μg/ml to about 20 μg/ml. The daily dosage may be administered in a form that contains one or more solubilizing agents, e.g., polyols.

Effective dosage amounts of the N-methylol transfer agents are provided in a composition may include dosage units containing about 0.01-500 mg/kg, about 1-100 mg/kg per day, or about 5-50 mg/kg per day of the N-methylol transfer agen. In some aspects, dosage units are administered every other day, biweekly, or weekly.

The specific effective dose for any particular patient will depend on a variety of factors including the severity or likelihood of the neovascularization and/or excessive angiogenesis, disorder or disease; activity of the specific compound employed; the age, body weight, general health, sex and diet of the patient; the preparation of the specific compound; the time and route of administration; the duration of administration; therapeutic agents used in combination or coinciding with the specific compound employed; and like factors known in the medical arts. The effective dose may also change over time as the GAPDH-mediated disorders, diseases, or conditions worsen or improve. For chronic conditions, subjects may receive effective doses for a plurality of days, weeks, months, years, or for the subject's lifetime. The number of and frequency of administrations or co-administrations may vary depending upon the likelihood or severity of the GAPDH-mediated disorder, disease or condition, and the patient specific response to the particular compound administered and/or a second therapeutically active agent that is administered to the subject.

In another aspect, the present disclosure provides a method, kit, apparatus or device for screening assay to identify additional inhibitors of GAPDH. One or more test compounds may be assayed for binding to and inhibition of GAPDH. In one aspect, the present disclosure involves combining a test compound with a suitable buffer or solvent, e.g., a buffer or solvent that dissolves the test compound, contacting the test compound with recombinant GAPDH in buffer to form a reaction mixture, and subjecting aliquots of the reaction mixture to an enzyme activity assay to identify compounds that inhibit GAPDH.

In some aspects, the enzyme activity assay may be performed in a multi-well plate and using a recombinant GAPDH probe to detect a change in NAD+ concentration compared to a control solvent. In some aspects, the enzyme activity assay may include sodium pyrophosphate buffer. In some aspects, recombinant GAPDH probe may be incubated with sodium arsenate, NAD+, and glyceraldehyde-3-phosphate (G3P). Enzyme activity may be measured using a microplate-reader spectrophotometer as the increase in absorbance at 340 nm due to reduction of NAD+, e.g., at room temperature. In some aspects, the recombinant GAPDH may first be diluted into sodium pyrophosphate buffer, e.g., to a volume of 100 μl. Subsequently, an additional 100 μl of reaction mix containing the sodium arsenate, NAD+, and G3P may be rapidly added to each well using a repeat pipettor, the plate may be mixed, e.g., for 5 seconds in the plate reader, and absorbance measurements are then taken. In some aspects, absorbance may be measured every 10-20 seconds for 20 minutes, and the rate is calculated from the change in absorbance during the linear phase. Inhibition of GAPDH is indicated by a reduction in the rate of reducing NAD+ compared to the control solvent.

In one aspect, the present disclosure provides a method of identifying inhibitors of GAPDH comprising combining a test compound with a solvent to form a solution, contacting the solution with recombinant GAPDH in buffer to form a reaction mixture, and subjecting aliquots of the reaction mixture to an enzyme activity assay, detecting change in NAD+ concentration in the enzyme activity assay, identifying a test compound that inhibit GAPDH by identifying a test compound that reduces NAD+ concentration in the enzyme activity assay compared to a control solvent.

In another aspect, the present disclosure provides a method, kit, apparatus or device for providing a biomarker for clinical use. In some aspects, the present disclosure provides a biomarker for use in patients suffering from or at risk of suffering from cancer. In some aspects, the present disclosure provides a method of using GAPDH as a biomarker by obtaining peripheral blood mononuclear cells (PBMCs) from a subject, lysing the PBMCs, and monitoring GAPDH activity in the lysed PBMCs. In some aspects, the method includes subjecting PBMCs lysates to an enzyme activity assay, detecting change in NAD+ concentration in the enzyme activity assay, and monitoring inhibition of GAPDH by an administered GAPDH-inhibitor based on reduction of NAD+ concentration in the enzyme activity assay compared to a control solvent.

Inhibition of GAPDH in peripheral blood mononuclear cells (PBMCs) may serve as a biomarker for the status of GAPDH inhibition in cancerous tissue. Similar to cancer tissue, the compounds of the present disclosure may inhibit GAPDH covalently in PMBCs. However, in contrast to cancer cells, GAPDH is not rate limiting in PBMCs and will not be deleterious to these cells. It is assumed that the half-life of the GAPDH protein in PMBCs is the same or similar to that in the patient's cancer tissue. The degree of inhibition of GAPDH in PMBCs may directly reflect the activity status of GAPDH in the target tissue.

In some aspects, the present disclosure provides a method for following the degree of GAPDH inhibition in patients treated with one or more compounds of the present disclosure by obtaining peripheral blood mononuclear cells (PBMCs) from a subject, lysing the PBMCs, monitoring GAPDH activity in the lysed PBMCs, subjecting the lysed PBMCs to an enzyme activity assay, detecting changes in NAD+ concentration in the enzyme activity assay, monitoring inhibition of GAPDH by an administered GAPDH-inhibitor based on reduction of NAD+ concentration in the enzyme activity assay compared to a control solvent, determining the degree of inhibition of GAPDH in the PBMCs, and identifying a subject as a suitable candidate for treating with a specific GAPDH-inhibitor compound of the present disclosure if the degree of inhibition of GAPDH by the specific compound is greater than a predetermined threshold, e.g., about 50%, about 60%, about 70%, about 80%, about 90%, or about 95%.

### EXAMPLES

Aspects of the present disclosure will be further described with reference to the following Examples, which are provided for illustrative purposes only and should not be used to limit the scope of or construe the invention.

### Example 1

Various N-methylol transfer agents of the present disclosure are synthesized and are assayed for interactions with GAPDH. The covalently labeled enzyme is purified and reactive intermediates are identified using various analytical methods including mass spectrometry of the labeled peptide is elucidated.

### Example 2

Inhibition of the LPS-stimulated cytokine release by compounds of the present disclosure is assayed and is found to be higher under high glucose (10 mM) versus low glucose (0.5 mM). Heptelidic acid is a positive control.

### Example 3

Using lactate production as a proxy measure, the impact of compounds of the present disclosure on the LPS stimulation is accompanied by a reduction in lactate. The LPS stimulation generates a Warburg-like increase in glycolysis. GAPDH becomes rate limiting only under such high glycolysis conditions.

### Example E 4

Recombinant GAPDH is directly inhibited bycompounds of the present disclosure. The incubation time may be critical as it is a cell-free assay. Specific doses required for a half-maximal or full inhibition of GAPDH are tested in vitro and in vivo in rodents. These in vitro and in vivo data provide a target-related measure of the dose required to inhibit the GAPDH in tissue, e.g., cancer tissue, to various degrees, which is a more direct measure of the impact of the compounds of the present disclosure in contrast to cellular assays such as induction of apoptosis or ROS production.

The degree of occupancy of GAPDH by the compounds of the present disclosure in patients are directly detected using a PET-compatible derivative of the compounds of the present disclosure, e.g., by incorporating Fluor 18.

### Example 5

GAPDH is the rate limiting glycolytic enzyme in cells operating under conditions of aerobic glycolysis, such as tumor cells. Partial inhibition is therefore expected to impair the energy metabolism of tumor cells. This is in contrast to normal cells. Their energy metabolism is largely based on oxidative phosphorylation. GAPDH is not a rate limiting enzyme of glycolysis in normal cells and a partial inhibition of GAPDH is tolerated. A test compound is dissolved in HEN buffer (250 mM Hepes-NaOH pH7.7, 1 mM EDTA, 0.1 mM neocuproine) or—for water-insoluble compounds—in DMSO (100 mM and is diluted with HEN buffer to the concentrations to be tested). Recombinant GAPDH (Abcam catalog number ab82633), 2 μg of enzyme, is diluted to 500 μl (final concentration˜110 nM) in HEN buffer and is treated with appropriate concentration of test compound at 37° C. At the indicated time points (30, 60, 180 minutes), aliquots are removed and subjected to the enzyme activity assay.

The enzyme activity assays are performed in 10 mM sodium pyrophosphate buffer (pH8.5) in 96-well plates. Recombinant GAPDH probe is incubated with 20 mM sodium arsenate (made fresh on day of experiment), 1 mM NAD+, and 2.88 mM glyceraldehyde-3-phosphate(G3P). Enzyme activity is measured using a microplate-reader spectrophotometer (Molecular Devices) as the increase in absorbance at 340 nm due to reduction of NAD+. The assay is performed at room temperature. The recombinant GAPDH is first diluted into sodium pyrophosphate buffer to a volume of 100 μl. An additional 100 μl of reaction mix containing the sodium arsenate, NAD+, and G3P is then rapidly added to each well using a repeat pipettor, the plate is mixed for 5 seconds in the plate reader, and measurements then begin. Absorbance is measured every 10-20 seconds for 20 minutes, and the rate is calculated from the change in absorbance during the linear phase. Inhibition of GAPDH is indicated by a reduction in the rate of reducing NAD+ compared to the control solvent.

While the subject matter of this disclosure has been described and shown in considerable detail with reference to certain illustrative examples, including various combinations and sub-combinations of features, those skilled in the art will readily appreciate other aspects and variations and modifications thereof as encompassed within the scope of the present disclosure. Moreover, the descriptions of such aspects, combinations, and sub-combinations is not intended to convey that the claimed subject matter requires features or combinations of features other than those expressly recited in the claims. Accordingly, the scope of this disclosure is intended to include all modifications and variations encompassed within the spirit and scope of the following appended claims.

What is claimed is:

1. A method of inhibiting GAPDH comprising administering to a subject in need of GAPDH-inhibition an N-methylol transfer agent.

2. A method of inhibiting GAPDH in a subject in need thereof comprising administering a composition comprising Taurolidine, Taurultam, 1183B (cyclo-taurolidine), N-methylol taurinamide, 1,3,-dimethylol-5,5-dimethylhydantoin, hexamethylene tetramine, or noxythiolin, N-methyltaurinamide, a substance which forms N-methylol taurinamide, a pharmaceutically acceptable salt, hydrate, ester, prodrug, or solvate thereof, or a combination thereof to the subject.

3. The method of claim 2, comprising inhibiting about 30%, about 40%, about 50%, about 60%, about 70%, about 80%, about 90%, or about 100% of GAPDH activity in cells of a subject comprising administering a composition comprising Taurolidine, Taurultam, 1183B (cyclo-taurolidine), N-methylol taurinamide, 1,3,-dimethylol-5,5-dimethylhydantoin, hexamethylene tetramine, or noxythiolin, N-methyltaurinamide, a substance which forms N-methylol taurinamide, a pharmaceutically acceptable salt, hydrate, ester, prodrug, or solvate thereof, or a combination thereof in an effective amount to the subject.

4. A method of reducing or inhibiting production of adenosine triphosphate (ATP) in a subject in need thereof comprising administering a composition comprising Taurolidine, Taurultam, 1183B (cyclo-taurolidine), N-methylol taurinamide, 1,3,-dimethylol-5,5-dimethylhydantoin, hexamethylene tetramine, or noxythiolin, N-methyltaurinamide, a substance which forms N-methylol taurinamide, a pharmaceutically acceptable salt, hydrate, ester, prodrug, or solvate thereof, or a combination thereof to the subject.

5. A method of preventing, inhibiting or reducing at least one sign or symptom of a disease, disorder or condition caused by or associated with GAPDH activity in a subject in need thereof comprising administering a composition comprising Taurolidine, Taurultam, 1183B (cyclo-taurolidine), N-methylol taurinamide, 1,3,-dimethylol-5,5-dimethylhydantoin, hexamethylene tetramine, or noxythiolin, N-methyltaurinamide, a substance which forms N-methylol taurinamide, a pharmaceutically acceptable salt, hydrate, ester, prodrug, or solvate thereof, or a combination thereof to the subject.

6. The method of claim 5, wherein the disease, disorder or condition is caused by or associated with impaired glycolysis, impaired protein degradation pathways, uncontrolled protein aggregation, aerobic glycolysis, mitochondrial dysfunction, increased glucose uptake or metabolism, neovascularization, autoimmune reactions, immune reactions, excessive angiogenesis, dysfunctional apoptosis of normal cells, impaired autophagy, or a combination thereof.

7. The method of claim 5, wherein the at least one sign or symptom is rash, acne, eczema, muscle pain, joint pain, fatigue, anemia, inflammation, abdominal pain, abdominal bloating, diarrhea, nausea, acid reflux, weight gain, fever, ongoing headaches, bleeding complications (e.g., hemorrhage), hypertension, hypotension, low blood counts, tumor-growth, cachexia, light sensitivity, eye redness, eye irritation, alopecia, loss of hair pigmentation, nail pitting, nail white spots, thick or rough nail folds, cuticles with spots or hyperpigmentation, spoon-shaped nails, thinning nails, onycholysis, splitting nails, shortness of breath, speech changes, ongoing headaches, pruritis, blurry vision, reduced balance, or a combination thereof.

8. A method of increasing production or localization of reactive species in a tumor of a subject in need thereof comprising administering a composition comprising Taurolidine, Taurultam, 1183B (cyclo-taurolidine), N-methylol taurinamide, 1,3,-dimethylol-5,5-dimethylhydantoin, hexamethylene tetramine, or noxythiolin, N-methyltaurinamide, a substance which forms N-methylol taurinamide, a pharmaceutically acceptable salt, hydrate, ester, prodrug, or solvate thereof, or a combination thereof to the subject.

9. (canceled)

10. (canceled)

11. The method of claim 2, wherein taurolidine is administered to the subject.

12. The method of claim 2, wherein the subject has suffered from or is suffering from a tumor, a cancer including, a skin disease, a diabetic ulcer, a chronic wound, a cardiovascular disease, stroke, a traumatic brain injury, macular degeneration, impaired glycolysis, impaired protein degradation pathways, uncontrolled protein aggregation, aerobic glycolysis, mitochondrial dysfunction, increased glucose uptake or metabolism, neovascularization, autoimmune reactions, immune reactions, excessive angiogenesis, dysfunctional apoptosis of normal cells, impaired autophagy, or a combination thereof.

13. (canceled)

14. (canceled)

15. A method of treating a subject suffering from a GAPDH-mediated disease, disorder, or condition comprising obtaining a biological sample comprising cells from a subject, lysing the cells, monitoring GAPDH activity in the lysed cells as a biomarker for GAPDH-mediated disease, and administering a composition comprising a GAPDH inhibitor to the subject.

16. The method of claim 15, wherein the cell lysates are subjected to an enzyme activity assay, changes in NAD+ concentration in the enzyme activity assay are detected, and inhibition of GAPDH by an administered GAPDH-inhibitor is monitored based on reduction of NAD+ concentration in the enzyme activity assay compared to a control solvent.

17. The method of claim 15, wherein the GAPDH inhibitor is a compound selected from Taurolidine, Taurultam, 1183B (cyclo-taurolidine), N-methylol taurinamide, 1,3,-dimethylol-5,5-dimethylhydantoin, hexamethylene tetramine, or noxythiolin, N-methyltaurinamide, a substance which forms N-methylol taurinamide, a pharmaceutically acceptable salt, hydrate, ester, prodrug, or solvate thereof, or a combination thereof.

18. The method of claim 15, wherein the cells are peripheral blood mononuclear cells (PBMCs).

19. The method of claim 15, wherein the method comprises analyzing the cells to determine the level of GAPDH inhibition over time.

20. (canceled)

21. (canceled)

22. (canceled)

23. A method of treating macular degeneration in a subject in need thereof comprising administering a composition comprising a compound selected from Taurolidine, Taurultam, 1183B (cyclo-taurolidine), N-methylol taurinamide, 1,3,-dimethylol-5,5-dimethylhydantoin, hexamethylene tetramine, or noxythiolin, N-methyltaurinamide, a substance which forms N-methylol taurinamide, a pharmaceutically acceptable salt, hydrate, ester, prodrug, or solvate thereof, or a combination thereof to the subject.

24. The method of claim 23, wherein the composition is an ophthalmic composition.

25. The method of claim 22, wherein the composition is administered by intravitreal injection.

26. Taurolidine, or a pharmaceutically acceptable salt, hydrate, ester, or solvate thereof.

27. (canceled)

28. (canceled)

29. (canceled)
